(19) United States
(12) Patent Application Publication
(10) Pub. No.: US 2001/0014357 A1
(43) Pub. Date: Aug. 16, 2001

(54) Citrus peel extract as inhibitor of ACYL coa-cholesterol-o-acyltransferase, inhibitor of macrophage-lipid complex accumulation on the arterial wall and preventive or treating agent for hepatic diseases

(75) Inventors: Song-Hae Bok, Daejeon (KR); Tae-Sook Jeong, Daejeon (KR); Ki-Hwan Bae, Daejeon (KR); Yong-Bok Park, Daegu (KR); Myung-Sook Choi, Daegu (KR); Surk-Sik Moon, Gongju-shi (KR); Yong-Kook Kwon, Daejeon (KR); Eun-Sook Lee, Daejeon (KR); Byung-Hwa Hyun, Daejeon (KR); Yang-Kyu Choi, Daejeon (KR); Chul-Ho Lee, Daejeon (KR); Jun-Sung Lee, Daejeon (KR); Kwang-Hee Son, Daejeon (KR); Byoung-Mog Kwon, Daejeon (KR); Young-Kook Kim, Daejeon (KR); Doil Choi, Daejeon (KR); Sung-Uk Kim, Daejeon (KR); Ingyu Hwang, Daejeon (KR); Jung-Ah Ahn, Daejeon (KR); Young-Bae Park, Seoul (KR); Hyo-Soo Kim, Seoul (KR); Seong-Choon Choe, Seoul (KR)

Correspondence Address: ANDERSON KILL & OLICK, P.C., 1251 Avenue of the Americas, New York, NY, 10020, US

(73) Assignee: KOREA INSTITUTE OF SCIENCE AND TECHNOLOGY

(21) Appl. No.: 09/728,917

(22) Filed: Feb. 12, 2001

Related U.S. Application Data
(62) Division of application No. 09/181,396, filed on Oct. 28, 1998, now abandoned.

(30) Foreign Application Priority Data
Oct. 28, 1997 (KR) 97-55580
Mar. 28, 1998 (KR) 98-10888
Apr. 1, 1998 (KR) 98-11450
Apr. 8, 1998 (KR) 98-12411
Apr. 14, 1998 (KR) 98-13283

(51) Int. Cl. A61K 35/78

(52) U.S. Cl. 424/736

(57) ABSTRACT

Methods for inhibiting the activity of acyl CoA-cholesterol-o-acyltransferase, inhibiting the accumulation of macrophage-lipid complex on the arterial endothelium, and preventing or treating hepatic diseases in a mammal comprise administering a citrus peel extract thereto.

### FIELD OF THE INVENTION

The present invention relates to methods for inhibiting the activity of acyl CoA-cholesterol-o-acyltransferase (ACAT), inhibiting the accumulation of macrophage-lipid complex on the arterial endothelium, and preventing or treating hepatic diseases in a mammal, said methods comprising administering a citrus peel extract to the mammal.

### BACKGROUND OF THE INVENTION

In recent years, coronary cardio-circulary diseases, e.g., atherosclerosis and hypercholesterolemia, have increasingly become a major cause of deaths. It has been reported that an elevated plasma cholesterol level causes the deposition of fat, macrophages and foam cells on the wall of blood vessels, such deposit leading to plaque formation and then to atherosclerosis(Ross, R., Nature, 362, 801-809(1993)). One of the methods for decreasing the plasma cholesterol level is alimentotherapy to reduce the ingestion of cholesterol and lipids. Another method is to inhibit the absorption of cholesterol by inhibiting enzymes involved therein.

Acyl CoA-cholesterol-o-acyltransferase(ACAT) promotes the esterification of cholesterol in blood. Foam cells are formed by the action of ACAT and contain a large amount of cholesterol ester carried by low density lipoproteins. The formation of foam cells on the wall of artery increases with the ACAT activity, and, accordingly, an inhibitor of ACAT may also be an agent for preventing atherosclerosis. Further, it has been reported that the blood level of LDL-cholesterol can be reduced by inhibiting the ACAT activity (Witiak, D. T. and D. R. Feller(eds.), Anti-Lipidemic Drugs: Medicinal, Chemical and Biochemical Aspects, Elsevier, ppl59-195 (1991)).

On the other hand, deterioration of hepatic functions may occur due to an excessive intake of alcohol or foods having a high lipid content, or an infection of hepatitis B or C virus, and it may develop into hepatitis, hepatocirrhosis or hepatic cancer. In particular, the excessive intake of fat-containing foods and alcohol causes fatty liver wherein a large amount of lipids is deposited in the liver tissue and the levels of serum GOT (glutamate-oxaloacetate transaminase), GPT (glutamate-pyruvate transaminase) and γ-GTP (γ-glutamyl transpeptidase) are elevated (T. Banciu et al., Med. Interne., 20, 69-71 (1982); and A. Par et al., Acta. Med. Acad. Sci. Huna., 33, 309-319 (1976)).

Numerous efforts have been made to develop medicines which inhibit ACAT activity; and, as a result, several compounds isolated from the cultures of various microorganisms have been reported. Examples of such compounds include pyripyropenes isolated from the culture of Aspergillus fumigatus (S. Omura et al., J. Antibiotics, 46, 1168-1169 (1993)) and Acaterin isolated from Pseudomonas sp. (S. Nagamura et al., J. Antibiotics, 45, 1216-1221 (1992)).

Further, as a treating agent for hypercholesterolemia, a HMG-CoA reductase inhibitor named Lovastatin® has been developed and marketed by Merck Co., U.S.A. However, this medicine is known to induce adverse side effect of increasing creatin kinase in the liver.

Accordingly, there has continued to exist a need to develop non-toxic inhibitors of ACAT and macrophage-lipid complex accumulation on the arterial epithelium, and a preventive or treating agent for the hepatic diseases.

The present inventors have endeavored to develop a novel and potent ACAT inhibitor, macrophage-lipid complex accumulation inhibitor and treating agent for the hepatic diseases from natural materials, and, as a result, have discovered that citrus peel extract has a potent ACAT inhibitory activity, macrophage-lipid complex accumulation inhibitory activity, and preventive or treating activity on the hepatic diseases.

Hitherto, citrus peel has been discarded or used only for the preparation of an animal fodder or organic fertilizer. Dried citrus peel comprises 50 to 60 wt % of alcohol-insoluble polymers such as pectin, hemicellulose and cellulose; 30 to 50 wt % of alcohol-soluble solid materials (80 wt % thereof consisting of glucose, fructose and sucrose); and a small or trace amount of bioflavonoids, vitamins, limonoids, phenolic compounds and oils. In particular, various bioflavonoids listed in Table I are present in the citrus peel(Horowitz, R. M., et al., J. Org. Chem. 25, 2183-2187 (1960)). Among the bioflavonoids, hesperidin is a major component of oranges, lemons and tangerines; naringin is a major component of grapefruits; and nearly the same amounts of naringin and hesperidin are present in citron.

TABLE I

| Citrus fruit | Bioflavonoids |
|---|---|
| Grapefruit | apigenin, dihydrokaempferol, eriodictyol, |
| | hesperetin, hesperidin, isorhamnetin, |
| | isosakuranetin, kaempferol, naringenin, |
| | naringin, neohesperidin, poncirin, |
| | quercetin, rutin |
| Lemon | apigenin, apigenin 7-rutinoside, |
| | chrysoeriol, diosmin, eriocitrin, |
| | hesperidin, isorhamnetin, limocitrin, |
| | limocitrol, luteolin 7-rutinoside, |
| | naringin, neohesperidin, poncirin, |
| | quercetin |
| Orange | auranetin, hesperidin, isosakuranetin 7- |
| | rutinoside, naringin, neohesperidin, |
| | nobiletin, rutin, sinensetin, tangeretin, |
| | vitexin |
| Tangerine | hesperidin, nobiletin, tangeretin |

It has been reported that the bioflavonoids isolated from a citrus peel have anti-oxidative, anti-cancer, anti-viral and blood-pressure lowering activities (Saija, A., et al., Free Radical Biol. Med., 19, 481-486 (1995); Matsubara, Y., et al., Japan Organic Synthesis Chem. Association Journal, 52, 318-327(1994, Mar.); Galati, E. M., et al., Farmaco., 51(3), 219-221 (1996, Mar.); Felicia, V., et al., Nutr. Cancer, 26, 167-181 (1996); EP 0352147 A2 (1990. 1. 24); and Kaul, T. N., et al., J. Med. Viol., 15, 71-75 (1985)). Further, limonoids present in the citrus peel have been reported to have an anti-cancer activity (Lam, L. K. T., et al., Inhibition of Chemically Induced Carcinogenesis by Citrus Limonoids, In Food Phytochemicals for Cancer Prevention, Vol. I, ACS Symposium series No. 546, M. T. Huang, O. Osawa, C. T. Ho, R. Rosen (eds), 1993).

However, hitherto, none of the ACAT inhibitory activity, macrophage-lipid complex accumulation inhibitory activity and preventive or treating activity on the hepatic diseases of the citrus peel extract has been reported.

### SUMMARY OF THE INVENTION

Accordingly, it is an object of the present invention to provide a method for inhibiting the ACAT activity in a mammal.

Another object of the present invention is to provide a method for inhibiting the accumulation of macrophage-lipid complex on the endothelial wall of an artery in a mammal.

A further object of the present invention is to provide a method for preventing or treating hepatic diseases in a mammal.

### BRIEF DESCRIPTION OF THE DRAWINGS

The above and other objects and features of the present invention will become apparent from the following description of the invention, when taken in conjunction with the accompanying drawings, in which:

FIGS. 1A, 1B, 1C and 1D show the arteries of the rabbits administered with 1% cholesterol; 1% cholesterol plus 1 mg/kg Lovastatine; 1% cholesterol plus 0.1% hesperidin; and 1% cholesterol plus 0.1% naringin, respectively; and

FIGS. 2A, 2B, 2C and 2D present the microscopic features of the livers of the rabbits administered with 1% cholesterol, 1% cholesterol plus 1 mg/kg Lovastatine; 1% cholesterol plus 0.1% hesperidin, and 1% cholesterol plus 0.1% naringin, respectively.

### DETAILED DESCRIPTION OF THE INVENTION

In accordance with one aspect of the present invention, there is provided a method for inhibiting the acyl-CoA cholesterol-o-acyltransferase(ACAT) activity in a mammal which comprises administering a citrus peel extract thereto.

In accordance with another aspect of the present invention, there is provided a method for inhibiting the accumulation of macrophage-lipid complex on the endothelial wall of an artery in a mammal which comprises administering a citrus peel extract thereto.

In accordance with a further aspect of the present invention, there is provided a method for preventing or treating hepatic diseases in a mammal which comprises administering a citrus peel extract thereto.

The citrus may be tangerines, oranges, lemons, grapefruits, citrons, and the like. It is preferable to use peel of citrus fruits produced by organic agricultural techniques without using chemical pesticides.

The citrus peel extract of the present invention may be prepared by any of the conventional methods using water or suitable solvents such as alcohols, Ca(OH)₂ and NaOH. For instance, 3 to 30 l of 20 to 95% ethanol is added to 1 kg of dried citrus peel and the mixture is allowed to stand at a temperature ranging from 25 to 80° C. for a period ranging from 1 to 12 hours. The resulting extract is filtered and the filtrate is concentrated, e.g., by vacuum, to obtain a concentrated peel extract. On the other hand, 5 to 30 l of 0.1 to 2% Ca(OH)₂ and NaOH is added to 1 kg of dried citrus peel and the mixture is allowed to stand at a temperature ranging from 25 to 60° C. for a period ranging from 1 to 5 hours. The resulting extract is filtered and the filtrate is adjusted to a pH ranging from 4.0 to 7.0 by adding 1N HCl thereto. The resulting filtrate is allowed to stand at a temperature ranging from 1 to 10° C. for a period ranging from 10 to 48 hours. The resulting precipitate is recovered and then dried to obtain a citrus peel extract.

Further, a citrus peel powder may be used in the present invention in place of the citrus peel extract. The citrus peel powder may be prepared by lyophilizing or drying the solid materials including citrus peel, which remains after squeezing juice from a citrus fruit, according to a conventional method and, then, powdering it to a particle size ranging from 50 to 250 μm.

The citrus peel extract exerts an inhibitory effect on the ACAT activity and the accumulation of macrophage-lipid complex on the endothelial wall of an artery, and a preventive or treating effect on hepatic diseases at a dose of 1.0 mg/kg/day or more, the inhibitory effect increasing with the dose thereof.

Moreover, in spite of its potent efficacies, the citrus peel extract shows little toxicity or mitogenicity in tests using mice. More specifically, the citrus peel extract exhibits no toxicity when it is orally administered to a mouse at a dose of 1,000 mg/kg, which corresponds to an oral administration dose of 50 to 100 g/kg body weight of citrus peel extract for a person weighing 50 kg. Further, the citrus peel extract exerts no adverse effects on the liver function.

The present invention also provides a pharmaceutical composition for inhibiting the ACAT activity and accumulation of macrophage-lipid complex on the endothelial wall of an artery, and for preventing or treating hepatic diseasse, which comprise a citrus peel extract as an active ingredient and pharmaceutically acceptable excipients, carriers or diluents.

A pharmaceutical formulation may be prepared in accordance with any of the conventional procedures. In preparing the formulation, the active ingredient is preferably admixed or diluted with a carrier, or enclosed within a carrier which may be in the form of a capsule, sachet or other container. When the carrier serves as a diluent, it may be a solid, semi-solid or liquid material acting as a vehicle, excipient or medium for the active ingredient. Thus, the formulations may be in the form of a tablet, pill, powder, sachet, elixir, suspension, emulsion, solution, syrup, aerosol, soft and hard gelatin capsule, sterile injectable solution, sterile packaged powder and the like.

Examples of suitable carriers, excipients, and diluents are lactose, dextrose, sucrose, sorbitol, mannitol, starches, gum acacia, alginates, gelatin, calcium phosphate, calcium silicate, cellulose, methyl cellulose, microcrystalline cellulose, polyvinylpyrrolidone, water, methylhydroxybenzoates, propylhydroxybenzoates, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a mammal by employing any of the procedures well known in the art.

The pharmaceutical composition of the present invention can be administered via various routes including oral, transdermal, subcutaneous, intravenous and intramuscular introduction. In case of human, a typical daily dose of the citrus peel extract may range from about 1 to 1,000 mg/kg body weight, preferably 10 to 500 mg/kg body weight, and can be administered in a single dose or in divided doses.

However, it should be understood that the amount of the active ingredient actually administered ought to be determined in light of various relevant factors including the condition to be treated, the chosen route of administration, the age, sex and body weight of the individual patient, and the severity of the patient's symptom; and, therefore, the above dose should not be intended to limit the scope of the invention in any way.

Moreover, the citrus peel extract can be incorporated in foods or beverages, as an additive or a dietary supplement, for the purpose of inhibiting the ACAT activity, inhibiting the accumulation of macrophage-lipid complex on the arterial endothelium and/or preventing or treating hepatic diseases. The foods or beverages may include meats; juices such as a vegetable juice(e.g., carrot juice and tomato juice) and a fruit juice(e.g., orange juice, grape juice, pineapple juice, apple juice and banana juice); chocolates; snacks; confectionery; pizza; foods made from cereal flour such as breads, cakes, crackers, cookies, biscuits, noodles and the likes; gums; dairy products such as milk, cheese, yogurt and ice creams; soups; broths; pastes, ketchups and sauces; teas; alcoholic beverages; carbonated beverages such as Coca-Colas and Pepsi-Colas; vitamin complexes; and various health foods.

In this case, the content of the citrus peel extract in a food or beverage may range from 0.5 to 10% by weight. In particular, the beverage according to the present invention may comprise 10 to 100 g of the citrus peel extract per 1000 ml of the beverage. In case of citrus peel powder, the content thereof in a food or beverage may range from 0.5 to 30% by weight.

As described above, the citrus peel extract can be used as an effective, non-toxic pharmaceutical agent for inhibiting ACAT activity, inhibiting the accumulation of macrophage-lipid complex on the arterial endothelium, and/or preventing or treating hepatic diseases.

The following Examples are intended to further illustrate the present invention without limiting its scope.

Further, percentages given below for solid in solid mixture, liquid in liquid, and solid in liquid are on a wt/wt, vol/vol and wt/vol basis, respectively, and all the reactions were carried out at room temperature, unless specifically indicated otherwise.

### EXAMPLE 1

### Preparation and Analysis of Citrus Peel Extract

The peels of tangerines(Cheju Island, Korea), citrons (Jeollanamdo, Korea), and oranges, grapefruits and lemons (California, Calif., U.S.A.) were dried at a room temperature and powdered to a particle size ranging from 100 to 200 μm. 50 ml of methanol was added to 500 mg each of the citrus peel powder and extracted in a water bath at 50° C. for 6 hours. The extract thus obtained was cooled and filtered, and then methanol was added to the filtrate to a volume of 50 ml.

To confirm the composition of the citrus peel extract obtained above, 5.0 μl of the resulting extract was subjected to high performance liquid chromatography (HPLC) using Lichrosorb RP-8 column (5 μm, 4×250 mm) which was pre-equilibrated with 37 % methanol and maintained at a temperature of 30° C. The extract was eluted with 37% methanol at a flow rate of 1.0 ml/min. Standard solutions were prepared by dissolving hesperidin and naringin (Sigma Chemical Co. U.S.A.) in methanol to final concentrations of 0.1, 0.2, 0.3, 0.4 and 0.5 mg/ml, respectively, and subjected to HPLC under the same condition as above. The eluates were detected at 280 nm with UV-VIS spectrophotometer and the contents of hesperidin and naringin were calculated by comparing the areas of HPLC profiles of the citrus peel extract and the standard solution. The contents (%) of hesperidin and naringin in various citrus peel extracts are shown in Table II.

TABLE II

| | Hesperidin (%) | Naringin (%) |
|---|---|---|
| Orange | 2.10 | trace amount |
| Lemon | 1.40 | trace amount |
| Tangerine | 2.10 | trace amount |
| grapefruit | — | 4.70 |
| citron | 0.80 | 0.80 |

### EXAMPLE 2

### Preparation of Citrus Peel Extract

(1) Method using Ethanol

The peel of tangerine (Cheju island, Korea) was dried at a room temperature and 5 l of 30% ethanol was added to 500 g of the dried peel. The peel was extracted at 60° C. for 5 hours. The extract thus obtained was filtered through cotton cloths and the filtrate was concentrated under vacuum to obtain 190 g of syrupy extract. The content of hesperidin in the citrus peel extract were examined in accordance with the method of Example 1 and it was discovered that the citrus peel extract contains 5.1 g of hesperidin.

Further, the composition of the citrus peel extract was confirmed by HPLC and the result is shown in Table III.

TABLE III

| Ingredient | Content (%) |
|---|---|
| Moisture | 65 |
| Free saccharide | |
| Fructose | 11 |
| Glucose | 11 |
| Sucrose | 6 |
| Hesperidin | 2.7 |
| Others | 4.3 |

(2) Method using Ca(OH)₂

The peel of tangerine(Cheju island, Korea) was dried at a room temperature and 5 l of 0.5 % Ca(OH)₂ solution was added to 500 g of the dried peel. The peel was extracted at a room temperature for 1 hour while stirring and the extract thus obtained was filtered through cotton cloths. 1N HCl solution was added to the filtrate to adjust its pH to 4.5. The same procedure as above was repeated to obtain a filtrate except that pH of the filtrate was adjusted to pH 6.8. The filtrates thus obtained were allowed to stand at 5° C. for 24 hours. The precipitates thus obtained were recovered and dried to obtain 5 g and 10 g of powders, respectively. HPLC analysis of the powers demonstrated that the citrus peel extracts contained 3.2 g and 6.55 g of hesperidin(purity: 64% and 65%), respectively.

(3) Method using NaOH

The peel of tangerine (Cheju island, Korea) was dried at a room temperature and 5 l of 0.5% NaOH was added to 500 g of the dried peel. The peel was extracted at a room temperature for 1 hour while stirring and the extract thus obtained was filtered through cotton cloths. 1 N HCl solution was added to the filtrate to adjust its pH to 4.5. The same procedure as above was repeated to obtain a filtrate except that pH of the filtrate was adjusted to pH 6.8. The filtrates thus obtained were allowed to stand at 5° C. for 24 hours. The precipitates thus obtained were recovered and dried to obtain 44 g and 49 g of powders, respectively. HPLC analysis of the powers demonstrated that the citrus peel extracts contained 13.9 g and 9.8 g of hesperidin (purity: 31% and 20%), respectively.

### EXAMPLE 3

### Toxicity of Orally Administered Citrus Peel Extract

7 to 8 week-old, specific pathogen-free ICR female mice (6 heads) each weighing about 25 to 29 g and male mice (6 heads) each weighing about 34 to 38 g were bred under a condition of temperature 22±1° C., moisture 55±5% and photoperiod 12L/12D. Fodder (Cheiljedang Co., mouse and rat fodder) and water were sterilized and fed to the mice.

The citrus peel extract obtained in Example 2(1) was dissolved in 0.5% Tween 80 to a concentration of 100 mg/ml, and the solution was orally administered to the mice in an amount of 0.2 ml per 20 g of mouse body weight. The solution was administered once and the mice were observed for 10 days for signs of adverse effects or death according to the following schedule: 1, 4, 8, and 12 hours after the administration and, every 12 hours thereafter. The weight changes of the mice were recorded every day to examine the effect of citrus peel extract. Further, on the 10th day, the mice were sacrificed and the internal organs were visually examined.

All the mice were alive at day 10 and the citrus peel extract showed no toxicity at a dose of 1,000 mg/kg. The autopsy revealed that the mice did not develop any pathological abnormality, and no weight loss was observed during the 10 day test period. Accordingly, it was concluded that the citrus peel extract is not toxic when orally administered to an animal.

### EXAMPLE 4

### Administration of Citrus Peel Extract to an Animal

20 four-week-old Sprague-Dawley rats (Taihan laboratory animal center, Korea) each weighing about 90 to 110 g were evenly divided into two dietary groups by a randomized block design. The rats of the two groups were fed with two different high-cholesterol diets, i.e., AIN-76 laboratory animal diet (ICN Biochemicals, Cleveland, Ohio, U.S.A.) containing 1% cholesterol(Control group), and 1% cholesterol plas 16.7% citrus peel extract obtained in Example 2(1), respectively. The compositions of diets fed to the two groups are shown in Table IV.

TABLE IV

Dietary group

| | Control | Citrus peel |
|---|---|---|
| Ingredients | group | extract*² group |
| Casein | 20 | 20 |
| D,L-methionine | 0.3 | 0.3 |
| Corn starch | 15 | 15 |
| Sucrose | 49 | 32.3 |
| Cellulose powder*¹ | 5 | 5 |
| Mineral mixture*¹ | 3.5 | 3.5 |
| Vitamin mixture*¹ | 1 | 1 |
| Choline bitartrate | 0.2 | 0.2 |
| Corn oil | 5 | 5 |
| Cholesterol | 1 | 1 |
| Citrus peel extract | — | 16.7 |
| Total | 100 | 100 |

*¹Purchased from TEKLAD premier Co. (Madison, WI, U.S.A.)
*²0.1% hesperidin equivalent

The rats were allowed to feed freely on the specified diet together with water for six weeks, the ingestion amount was recorded daily and the rats were weighed every 7 days, and then the record was analyzed. All rats showed a normal growth rate and there was observed no significant difference among the two groups in terms of the feed ingestion amount and the weight gain.

### EXAMPLE 5

### Determination of Total Cholesterol, HDL-Cholesterol and Neutral Lipid Content in Plasma

The effect of administering citrus peel extract to rats on the plasma cholesterol and neutral lipid content was determined as follows.

Blood samples were taken from the rats of the two dietary groups and plasma HDL fractions were separated therefrom by using HDL-cholesterol reagent (Sigma Chemical Co., Cat. No. 352-3) containing dextran-sulfate. Total cholesterol and HDL-cholesterol levels were determined by using Sigma Diagnostic Kit Cat. No. 352-100 (Sigma Chemical Co., U.S.A.)(Allain et al., Clin. Chem., 20, 470-475 (1974)). Neutral lipid level was determined by using Sigma Diagnostic Kit Cat. No. 339-50 (Bucolo, G. and David, H., Clin. Chem., 19, 476-482 (1973)). The result is shown in Table V, wherein the total plasma cholesterol level decreased by 36% in the citrus peel extract-fed rat group, as compared with that of the control group.

TABLE V

Group

| | Control | Citrus peel extract |
|---|---|---|
| Lipid Conc. | group | group |
| Total-C (mg/dl) | 147.8 ± 34.8 | 94.2 ± 23 |
| HDL-C (mg/dl) | 22.2 | 23.5 |
| $\frac{HDL \text{-} C}{Total \text{-} C} ( \% )$ | 15.7 ± 5.3 | 26.2 ± 7.5 |
| TG (mg/dl) | 99.2 ± 18.9 | 108.5 ± 15.9 |

* Total-C: Total-cholesterol
* HDL-C: HDL-cholesterol
* TG: Triglyceride

### EXAMPLE 6

### Activity of Citrus Peel Extract in ACAT Inhibition

(Step 1) Preparation of Microsomes

To determine the effect of citrus peel extract feeding to rats on the activity of ACAT, microsomes were separated from the liver tissue to be used as an enzyme source.

First, the rats of the two groups prepared in Example 4 were sacrificed by decapitation and the livers were excised. 1 g each of the livers was homogenized in 5 ml of homogenization medium (0.1 M KH₂PO₄, pH 7.4, 0.1 mM EDTA and 10 mM β-mercaptoethanol). The homogenate was centrifuged at 3,000×g for 10 min. at 4° C. and the supernatant thus obtained was centrifuged at 15,000×g for 15 min. at 4° C. to obtain a supernatant. The supernatant was put into an ultracentrifuge tube(Beckman) and centrifuged at 100,000×g for 1 hour at 4° C. to obtain microsomal pellets, which were then suspended in 3 ml of the homogenization medium and centrifuged at 100,000×g for 1 hour at 4° C. The pellets thus obtained were suspended in 1 ml of the homogenization medium. The concentration of proteins in the resulting suspension was determined by Lowry's method and then adjusted to 4 to 8 mg/ml. The resulting suspension was stored in a deep freezer (Biofreezer, Forma Scientific Inc.).

(Step 2) ACAT Assay

6.67 μl of 1 mg/ml cholesterol solution in acetone was mixed with 6 μl of 10% Triton WR-1339 (Sigma Co.) in acetone and, then, acetone was removed from the mixture by evaporation using nitrogen gas. Distilled water was added to the resulting mixture in an amount to adjust the concentration of cholesterol to 30 mg/ml.

To 10 μl of the resulting aqueous cholesterol solution were added 10 μl of 1 M KH₂PO₄(pH 7.4), 5 μl of 0.6 mM bovine serum albumin (BSA), 10 μl of microsome solution obtained in (Step 1) and 55 μl of distilled water (total 90 μl). The mixture was pre-incubated in a waterbath at 37° C. for 30 min.

10 μl of (1-¹⁴C) oleoyl-COA solution (0.05 μCi, final concentration: 10 μM) was added to the pre-incubated mixture and the resulting mixture was incubated in a waterbath at 37° C. for 30 min. To the mixture were added 500 μl of isopropanol:heptane mixture (4:1(v/v)), 300 μl of heptane and 200 μl of 0.1 M KH₂PO₄(pH 7.4), and the mixture was mixed violently by using a vortex and then allowed to stand at a room temperature for 2 min.

200 μl of the resulting supernatant was put in a scintillation bottle and 4 ml of scintillation fluid (Lumac) was added thereto. The mixture was assayed for radioactivity with 1450 Microbeta liquid scintillation counter (Wallacoy, Finland). ACAT activity was calculated as picomoles of cholesteryl oleate synthesized per min. per mg protein (pmoles/min/mg protein). The result is shown in Table VI.

TABLE VI

| | ACAT activity | % Inhibition on |
|---|---|---|
| Group | (pmole/min/mg protein) | ACAT activity |
| Control group | 806.2 ± 105.2 | 0 |
| Citrus peel | 548.0 ± 65.4 | 32 |
| extract group | | |

As can be seen from Table VI, ACAT activity observed in the citrus peel extract-fed rat group is lower than that of the control group by 32%.

### EXAMPLE 7

### Inhibition of Plaque Formation Caused by Macrophage-Lipid Complex in Citrus Peel Extract-Fed Animals

(Step 1) Admin.stration of citrus bioflavonoids to animals

36 three-month-old New Zealand White rabbits (Yeonam Horticulture and Animal Husbandry College, Korea) each weighing about 2.5 to 2.6 kg were bred under a condition of temperature 20±2° C., relative humidity 55±5%, and photoperiod 12L/12D. The rabbits were divided by a group of 6 rabbits, and the rats of six groups were fed with six different diets, i.e., RC4 diet (Oriental Yeast Co., Japan) containing 1% cholesterol(Control group); 1% cholesterol plus 1 mg/kg Lovastatin® (Merck, U.S.A.) (Comparative group); 1% cholesterol plus 0.1% hesperidin; 1% cholesterol plus 0.1% hesperetin; 1% cholesterol plus 0.1% naringin; and 1% cholesterol plus 0.1% naringenin, respectively. RC4 diet comprises 7.6% moisture, 22.8% crude protein, 2.8% crude fat, 8.8% crude ash, 14.4% crude cellulose and 43.6% soluble nitrogen-free substances. The rabbits were bred for 6 weeks while being allowed free access to the diets and water.

(Step 2) Analysis for Fatty Streak in the Main Artery

The rabbits bred in (Step 1) were sacrificed and their chest were incised. The main artery was cut out therefrom in a length of about 5 cm downward from the site 1 cm above the aortic valve and the fat surrounding the main artery was removed. The main artery was incised in the middle along the longitudinal axis and pinned to a dish. The moist artery was photographed and, then, staining of fatty streak was carried out in accordance with the method of Esper, E., et al.(J. Lab. Clin. Med., 121, 103-110 (1993)) as follows.

A part of the incised main artery was washed three times by 2 min. with anhydrous propylene glycol and stained for 30 min. with a saturated solution of Oil Red O (ORO, Sigma Co.) dissolved in propylene glycol. Thereafter, the artery was washed twice by 3 min. with 85% propylene glycol to remove remaining staining solution and, then washed with physical saline. The artery was photographed and the photograph was traced. The area of stained region (fatty streak region) was determined with an image analyzer (LEICA, Q-600, Germany) and its proportion(%) to the total arterial area was calculated.

On the other hand, the other part of the main artery was stained in accordance with hematoxylin-eosin (H&E) and Masson's trichrome staining methods and observed under a microscope to confirm whether the macrophage-lipid complexes were accumulated in the intima, internus, elastic lamina and media.

Further, blood samples were taken from the rabbits and total cholesterol and triglyceride levels were determined in accordance with the same procedure in Example 5.

The result is shown in Table VII.

TABLE VII

| | Total | | M-L* |
|---|---|---|---|
| | cholesterol | Triglyceride | complex area |
| Dietary Group | (mg/dl) | (mg/dl) | (%) |
| Control group | 1143 | 56 | 35 |
| 1 mg/kg Lovastatin | | | |
| group | 1210 | 66 | 5 |
| 0.1% hesperidin | 1130 | 40 | 13.5 |
| group | | | |
| 0.1% hesperetin | 1150 | 41 | 13 |
| group | | | |
| 0.1% naringin | 1367 | 72 | 12 |
| group | | | |
| 0.1% naringenin | 1350 | 70 | 13 |
| group | | | |

*M-L complex: Macrophage-lipid complex

As can be seen from Table VII, the area of macrophage-lipid complex accumulated on the arterial endothelium decreased significantly in the 1 mg/kg Lovastatin®, 0.1% hesperidin, 0.1% hesperetin, 0.1% naringin and 0.1% naringenin groups, as compared to the control group. Accordingly, it has been confirmed that hesperidin, hesperetin, naringin and naringenin isolated from citrus peel extract, as well as citrus peel extract containing the flavonoids, inhibit the accumulation of macrophage-lipid complex on the arterial endothelium. In particular, it is remarkable that the inhibitory activity of the biof lavonoids isolated from citrus peel extract on the accumulation of macrophage-lipid complex was exhibited under the blood cholesterol levels above 1,100 mg/dl, which are much higher than that of normal rabbit, i.e., about 50 mg/dl. This result suggests that there may be a novel mechanism for preventing the onset of atherosclerosis, which is different from the blocking of cholesterol synthesis by a HMG-CoA reductase inhibitor, blocking of cholesterol absorption by an ACAT inhibitor, or blocking of cholesterol transfer by a CETP inhibitor.

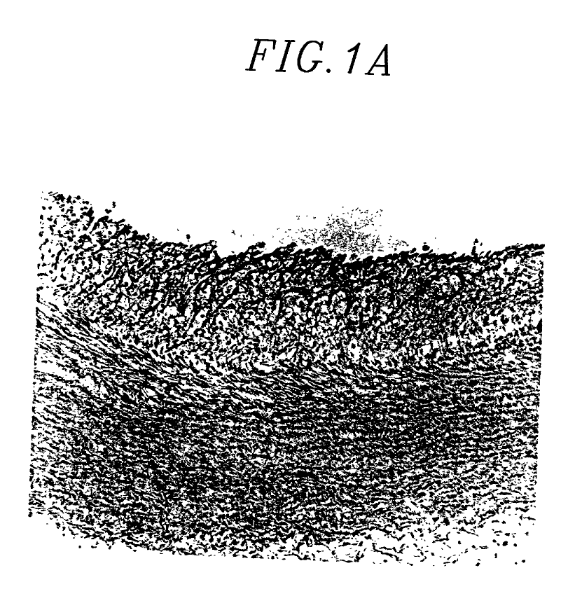

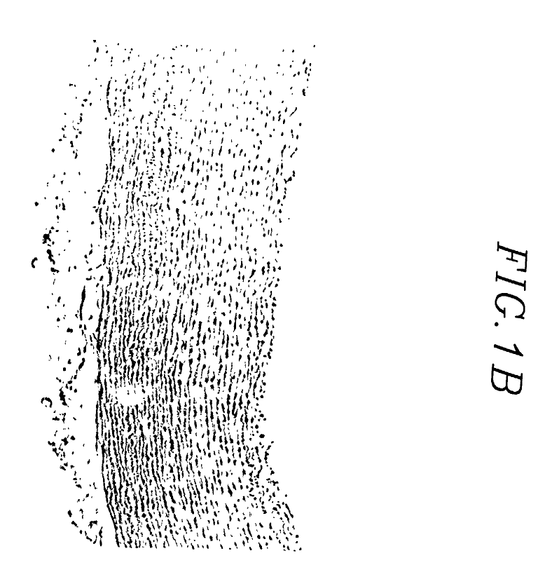

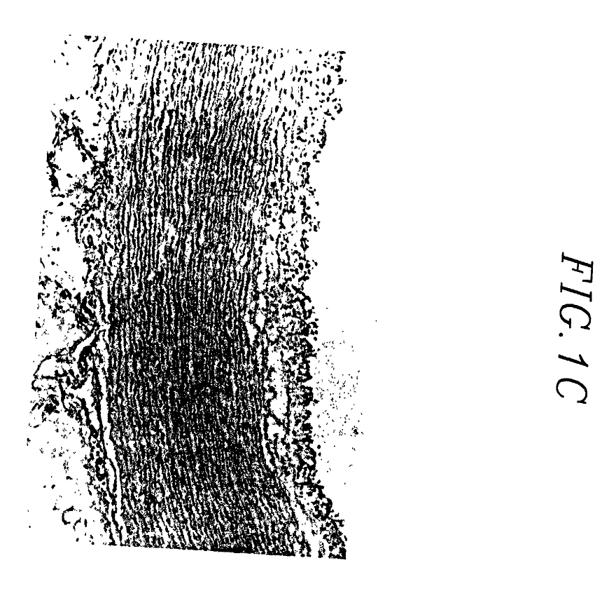

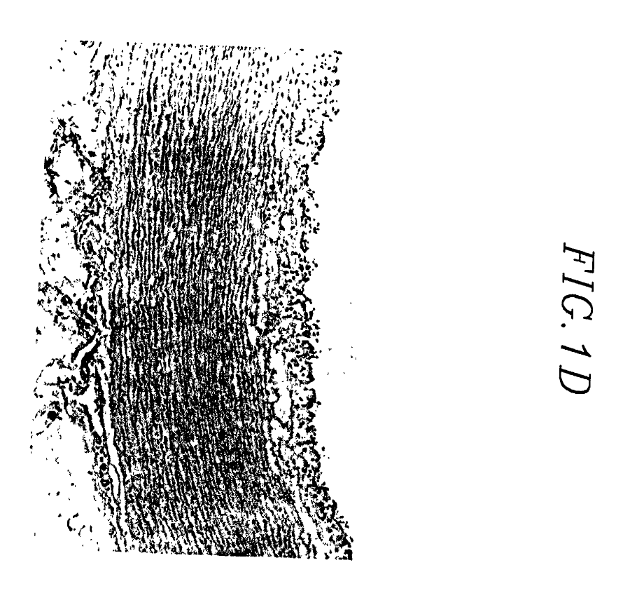

FIGS. 1A, 1B, 1C and 1D show the arteries of the rabbits administered with-1% cholesterol (control group); 1% cholesterol plus 1 mg/kg Lovastatin® (comparative group); 1% cholesterol plus 0.1% hesperidin; and 1% cholesterol plus 0.1% naringin, respectively. As shown in FIGS. 1A, 1B, 1C and 1D, a thick layer of macrophage-lipid complex was observed on the arterial endothelium of the rabbit administered with 1% cholesterol, while no or very thin layers of macrophage-lipid complex were observed on the arterial endotheliums of the rabbits administered with 1% cholesterol plus 1 mg/kg Lovastatin®, 1% cholesterol plus 0.1% hesperidin, and 1% cholesterol plus 0.1% naringin, respectively.

Accordingly, it has been concluded that citrus bioflavonoids such as hesperidin, hesperetin, naringin and naringenin, as well as citrus peel extract strongly inhibit the accumulation of macrophage-lipid complex on the arterial endothelium.

### EXAMPLE 8

### Prevention of Hepatic Diseases by Citrus Peel Extract

(Step 1) Administration of Citrus Peel Extract or Citrus Bioflavonoids to Rats

30 four-week-old Sprague-Dawley rats (Taihan laboratory animal center, Korea) each weighing about 90 to 110 g were evenly divided into three dietary groups by a randomized block design. The rats of the three groups were fed with three different high-cholesterol diets, i.e., AIN-76 laboratory animal diet (ICN Biochemicals, Cleveland, Ohio, U.S.A.) containing 1% cholesterol(Control group), 1% cholesterol plus 0.02% naringin, and 1% cholesterol plus citrus peel extract prepared in Example 2(1) in an amount equivalent to 0.04% hesperidin, respectively. The compositions of the diets fed to the three groups are shown in Table VIII.

TABLE VIII

| | Dietary group | | |
|---|---|---|---|
| | | 0.02% | Citrus peel |
| | Control | naringin | extract*³ |
| Ingredient | group | group | group |
| Casein | 20 | 20 | 20 |
| D,L-methionine | 0.3 | 0.3 | 0.3 |
| Corn starch | 15 | 15 | 15 |
| Sucrose | 39 | 38.98 | 22.5 |
| Cellulose powder*¹ | 5 | 5 | 5 |
| Mineral mixture*¹ | 3.5 | 3.5 | 3.5 |
| Vitamin mixture*¹ | 1 | 1 | 1 |
| Choline bitartrate | 0.2 | 0.2 | 0.2 |
| Fat | 15 | 15 | 15 |
| Cholesterol | 1 | 1 | 1 |
| Naringin*² | — | 0.02 | — |
| Citrus peel extract*³ | — | — | 16.5 |
| Total | 100 | 100 | 100 |

*¹Purchased from TEKLAD premier Co. (Madison, WI, U.S.A.)
*²Purchased from Sigma Chemical Co. (St. Louis, Mo, U.S.A.)
*³0.04% hesperidin equivalent

The rats were allowed to feed freely on the specified diet together with water for six weeks, the ingestion amount was recorded daily and the rats were weighed every 7 days, and then the record was analyzed. All rats showed a normal growth rate and there was observed no significant difference among the three groups in terms of the feed ingestion amount and the weight gain.

(Step 2) Determination of Serum GOT and GPT Levels

The effect of administering naringin and citrus peel extract to rats on the function of the liver was examined as follows.

Blood samples were taken from the rats of the three dietary groups and serum GOT (glutamate-oxaloacetate transaminase) and GPT (glutamate-pyruvate transaminase) levels were determined in accordance with the method of Reitman and Frankel (Reitman, S. and J. S. Frankel, Am. J. Clin. Pathol., 28, 56 (1956)). GOT and GPT are synthesized in the liver and heart, and released into blood stream upon the damage of these organs. Accordingly, GOT and GPT are representative markers of the liver-function and high serum GOT and GPT levels mean severe damage of the liver.

The result showed that GOT and GPT levels of citrus extract group and naringin group were lower than those of the control group by about 30% and 10%, respectively.

(Step 3) Experiment Using Rabbits

The same procedure as in (Step 1) was repeated except that 40 three-month-old New Zealand White rabbits (Yeonam Horticulture and Animal Husbandry College, Korea) each weighing about 2.5 to 2.6 kg were used in place of the rats, and the rabbits were fed for eight weeks with four different diets, i.e., RC4 diet containing 1% cholesterol (Control group); 1% cholesterol plus 1 mg/kg Lovastatin® (Comparative group); 1% cholesterol plus 0.1% hesperidin; and 1% cholesterol plus 0.1% naringin, respectively.

Thereafter, the livers were separated from the rabbits and the histopathological observations were carried out as follows.

The rabbits were anesthetized with an intramuscular injection of ketamine (75 mg/kg) and subjected to an abdominal incision. The color and degree of sclerosis of the liver were observed with eyes, and the liver separated from the rabbit was fixed in 10% neutral buffered formalin for more than 24 hours. The fixed liver was washed sufficiently with water, dehydrated stepwise with 70%, 80%, 90% and 100% ethanol and, then, embedded in paraffin. The embedded liver was sectioned in 4 μm thickness with a microtome and stained with hematoxylin and eosin. The stained liver specimen was made transparent with xylene, mounted with permount, and then observed under a microscope to confirm the presence of lesions.

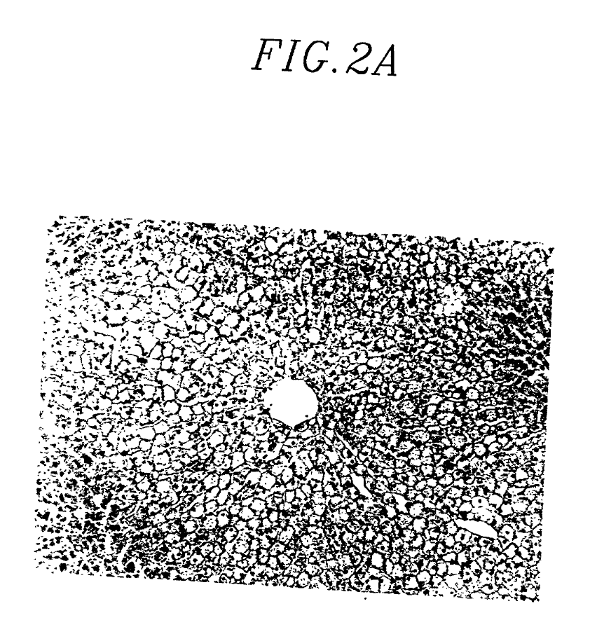

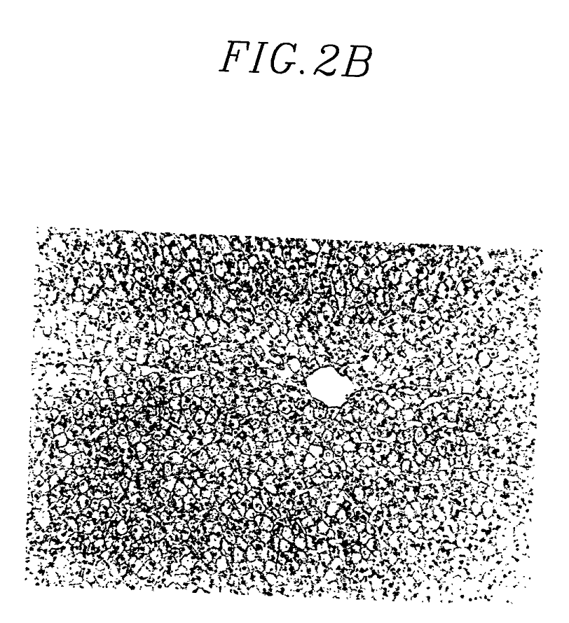

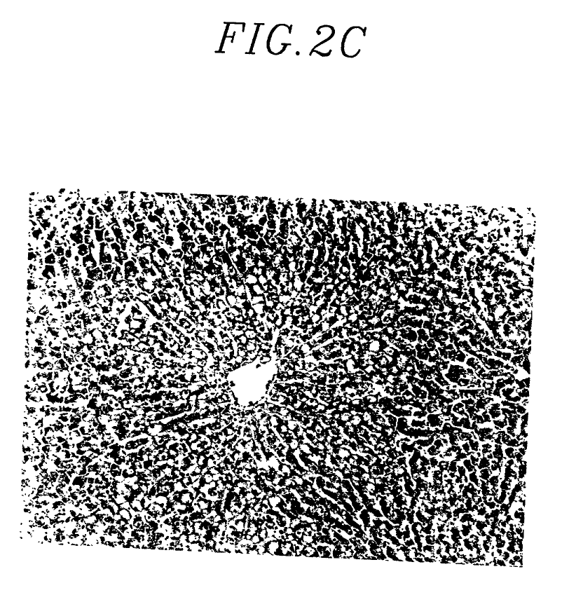

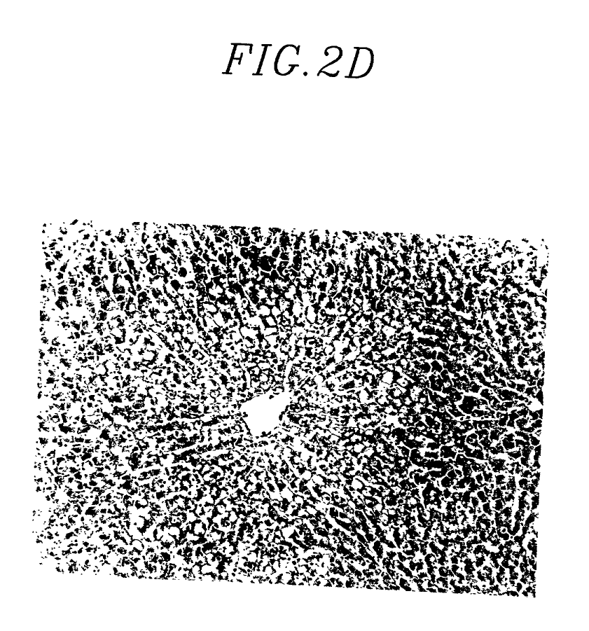

FIGS. 2A, 2B, 2C and 2D present the microscopic features of the livers of the rabbits administered with 1% cholesterol(control group), 1% cholesterol plus 1 mg/kg Lovastatin® (comparative group), 1% cholesterol plus 0.1% hesperidin, and 1% cholesterol plus 0.1% naringin, respectively. As shown in FIGS. 2A and 2B, the hepatic cells of the control group and the comparative group are irregularly arranged and enlarged and a large amount of fat is deposited therein. In contrast, as shown in FIGS. 2C and 2D, the hepatic cells of the hesperidin and naringin groups are normal and the deposition of fat is not observed. This result shows that the citrus biof lavonoid, i.e., hesperidin and naringin, and the citrus peel extract containing them strongly inhibit the occurrence of fatty liver without toxic adverse effect to the hepatic cells.

(Step 4) Experiment Using Human

Naringin was orally administered to a 55-year-old man at a daily dose of 10 mg/kg for 68 days and serum GOT, GPT and γGTP levels were determined just before the administration (day 0), and 45 and 68 days after the administration (day 45 and day 68), respectively. Consequently, serum GOT- levels at day 45 and day 68 decreased by 17%, respectively, in comparison to that of day 0. Serum GPT levels at day 45 and day 68 decreased by 15% and 19%, respectively, in comparison to that of day 0. Further, serum γGTP levels at day 45 and day 68 decreased by 25% and 51%, respectively, in comparison to that of day 0. Surprisingly, reduction of serum γGTP level at day 68 was more than 50%, and this result suggests that naringin and citrus peel extract containing it have a strong liver-protective activity and preventive activity on the hepatic diseases such as hepatitis, fatty liver and alcoholic fatty liver.

On the other hand, naringin was orally administered to a 56-year-old man, who had drunk alcoholic beverages habitually in an amount of 100 cc per day, at a daily dose of 6 mg/kg for 30 days and serum γGTP level was determined just before the administration (day 0) and 30 days after the administration (day 30). Consequently, initial serum γGTP level at day 0 was 129 IU/l, while that of day 30 decreased to 69 IU/l which is within the normal range. This result demonstrates that naringin or citrus peel extract containing it has a high activity of preventing alcoholic fatty liver and hepatocirrhosis.

### EXAMPLE 9

### Foods Containing Citrus Peel Powder or Extract

Foods containing citrus peel powder or extract obtained in Examples 1 and 2 were prepared as follows.

(1) Preparation of Tomato Ketchup and Sauce

The citrus peel powder obtained in Example 1 was added to a tomato ketchup or sauce in an amount ranging from 1 to 20 wt % to obtain a health-improving tomato ketchup or sauce.

Alternatively, the citrus peel extract obtained in Example 2(1) was added to a tomato ketchup or sauce in an amount ranging from 0.5 to 10 wt % to obtain a health-improving tomato ketchup or sauce.

(2) Preparation of Wheat Flour Foods

The citrus peel powder obtained in Example 1 was added to a wheat flour in an amount ranging from 1 to 30 wt % and breads, cakes, cookies, crackers and noodles were prepared by using the mixture to obtain health-improving foods.

Alternatively, these foods were prepared by using a wheat flour containing 0.5 to 10 wt % of the citrus peel extract obtained in Example 2(1).

(3) Preparation of Soups and Gravies

The citrus peel powder obtained in Example 1 was added to soups and gravies in an amount ranging from 1 to 30 wt % to obtain health-improving soups and gravies.

Alternatively, these foods were prepared by using soups and gravies containing 0.5 to 10 wt % of the citrus peel extract obtained in Example 2(1).

(4) Preparation of Ground Beef

The citrus peel powder obtained in Example 1 was added to ground beef in an amount ranging from 1 to 30 wt % to obtain a health-improving ground beef.

Alternatively, these foods were prepared by using ground beef containing 0.5 to 10 wt % of the citrus peel extract obtained in Example 2(1).

(5) Preparation of Dairy Product

The citrus peel powder obtained in Example 1 or citrus peel extract obtained in Example 2(1) was added to milk in an amount ranging from 0.5 to 10 wt % and various dairy products such as butter and ice cream were prepared by using the milk.

However, in case of cheese preparation, the citrus peel powder or extract was added to the coagulated milk protein; and, in case of yogurt preparation, the citrus peel powder or extract was added to the coagulated milk protein obtained after the fermentation.

### EXAMPLE 10

### Beverages containing Citrus Peel Powder or Extract

(1) Preparation of Vegetable Juice

10 to 100 g of the citrus peel powder obtained in Example 1 or citrus peel extract obtained in Example 2(1) was added to 1000 ml of a tomato or carrot Juice to obtain a health-improving vegetable juice.

(2) Preparation of Fruit Juice

10 to 100 g of the citrus peel powder obtained in Example 1 or citrus peel extract obtained in Example 2(1) was added to 1000 ml of an apple or grape Juice to obtain a health-improving fruit juice.

(3) Preparation of Carbonated Drink

1 to 100 g of the citrus peel powder obtained in Example 1 or citrus peel extract obtained in Example 2(1) was added to 1000 ml of Coca-Cola® or Pepsi-Cola® to obtain a health-improving carbonated drink.

While the invention has been described with respect to the above specific embodiments, it should be recognized that various modifications and changes may be made to the invention by those skilled in the art which also fall within the scope of the invention as defined by the appended claims.

What is claimed is:

1. A method for inhibiting the activity of acyl CoA-cholesterol-o-acyltransferase (ACAT) in a mammal which comprises administering an effective amount of citrus peel extract or citrus peel powder thereto.

2. The method of claim 1, wherein the mammal is human.

3. The method of claim 2, wherein the effective amount of the citrus peel extract ranges from 1 to 1,000 mg/kg body weight/day.

4. The method of claim 2, wherein the effective amount of the citrus peel powder ranges from 1 to 1,000 mg/kg body weight/day.

5. The method of claim 1, wherein the citrus is tangerines, oranges, lemons or grapefruits.

6. The method of claim 1, wherein the citrus peel extract is prepared by a process including the steps of: adding 3 to 30 l of 20 to 95% ethanol to 1 kg of dried citrus peel; allowing the mixture to stand at a temperature ranging from 25 to 80° C. for a period ranging from 1 to 12 hours; filtering the resulting extract; and concentrating the filtrate to obtain the citrus peel extract.

7. The method of claim 1, wherein the citrus peel extract is prepared by a process including the steps of: adding 5 to 30 l of 0.1 to 2% Ca(OH)₂ or NaOH to 1 kg of dried citrus peel; allowing the mixture to stand at a temperature ranging from 25 to 60° C. for a period ranging from 1 to 5 hours; filtering the resulting extract; adjusting the filtrate to a pH ranging from 4.0 to 7.0; allowing the resulting filtrate to stand at a temperature ranging from 1 to 10° C. for a period ranging from 10 to 48 hours; and, recovering and drying the resulting precipitate to obtain the citrus peel extract.

8. The method of claim 1, wherein the citrus peel powder is prepared by a process including the steps of: lyophilizing or drying the solid materials remaining after squeezing juice from citrus fruits; and powdering the dried materials to a particle size ranging from 50 to 250 μm.

9. The method of claim 1, wherein the citrus peel extract or citrus peel powder is administered in the form of a pharmaceutical composition containing an effective amount of the citrus peel extract and a pharmaceutically acceptable carrier.

10. The method of claim 1, wherein the citrus peel extract or citrus peel powder is administered in the form of an additive or a dietary supplement in food or beverage.

11. The method of claim 10, wherein the content of the citrus peel extract in the food ranges from 0.5 to 10% by weight.

12. The method of claim 10, wherein the content of the citrus peel powder in the food ranges from 1 to 30% by weight.

13. The method of claim 10, wherein the food is meats, chocolates, snacks, confectionery, pizza, foods made from cereal flour, gums, dairy products, soups, broths, pastes, ketchups, sauces, vitamin complexes or health foods.

14. The method of claim 13, wherein the foods made from cereal flour is breads, cakes, crackers, cookies, biscuits or noodles.

15. The method of claim 10, wherein the beverage is dairy products, vegetable juices, fruit juices, teas, alcoholic beverages or carbonated beverages.

16. The method of claim 10, wherein the content of the citrus peel extract in the beverage ranges from 10 to 100 g per 1,000 ml of the beverage.

17. A method for inhibiting the accumulation of macrophage-lipid complex on the arterial endothelium in a mammal which comprises administering an effective amount of citrus peel extract thereto.

18. The method of claim 17, wherein the mammal is human.

19. The method of claim 18, wherein the effective amount of the citrus peel extract ranges from 1 to 1,000 mg/kg body weight/day.

20. The method of claim 18, wherein the effective amount of the citrus peel powder ranges from 1 to 1,000 mg/kg body weight/day.

21. The method of claim 17, wherein the citrus is tangerines, oranges, lemons or grapefruits.

22. The method of claim 17, wherein the citrus peel extract is prepared by a process including the steps of: adding 3 to 30 l of 20 to 95% ethanol to 1 kg of dried citrus peel; allowing the mixture to stand at a temperature ranging from 25 to 80° C. for a period ranging from 1 to 12 hours; filtering the resulting extract; and concentrating the filtrate to obtain the citrus peel extract.

23. The method of claim 17, wherein the citrus peel extract is prepared by a process including the steps of: adding 5 to 30 l of 0.1 to 2% Ca(OH)₂ or NaOH to 1 kg of dried citrus peel; allowing the mixture to stand at a temperature ranging from 25 to 60° C. for a period ranging from 1 to 5 hours; filtering the resulting extract; adjusting the filtrate to a pH ranging from 4.0 to 7.0; allowing the resulting filtrate to stand at a temperature ranging from 1 to 10° C. for a period ranging from 10 to 48 hours; and, recovering and drying the resulting precipitate to obtain the citrus peel extract.

24. The method of claim 17, wherein the citrus peel powder is prepared by a process including the steps of: lyophilizing or drying the solid materials remaining after squeezing juice from citrus fruits; and powdering the dried materials to a particle size ranging from 50 to 250 μm.

25. The method of claim 17, wherein the citrus peel extract or citrus peel powder is administered in the form of a pharmaceutical composition containing an ef fective amount of the citrus peel extract and a pharmaceutically acceptable carrier.

26. The method of claim 17, wherein the citrus peel extract or citrus peel powder is administered in the form of an additive or a dietary supplement in food or beverage.

27. The method of claim 26, wherein the content of the citrus peel extract in the food ranges from 0.5 to 10% by weight.

28. The method of claim 26, wherein the content of the citrus peel powder in the food ranges from 1 to 30% by weight.

29. The method of claim 26, wherein the food is meats, chocolates, snacks, confectionery, pizza, foods made from cereal flour, gums, dairy products, soups, broths, pastes, ketchups, sauces, vitamin complexes or health foods.

30. The method of claim 29, wherein the foods made from cereal flour is breads, cakes, crackers, cookies, biscuits or noodles.

31. The method of claim 26, wherein the beverage is dairy products, vegetable juices, fruit juices, teas, alcoholic beverages or carbonated beverages.

32. The method of claim 26, wherein the content of the citrus peel extract in the beverage ranges from 10 to 100 g per 1,000 ml of the beverage.

33. A method for preventing or treating a hepatic disease in a mammal which comprises administering an effective amount of citrus peel extract thereto.

34. The method of claim 33, wherein the mammal is human.

35. The method of claim 34, wherein the effective amount of the citrus peel extract ranges from 1 to 1,000 mg/kg body weight/day.

36. The method of claim 34, wherein the effective amount of the citrus peel powder ranges from 1 to 1,000 mg/kg body weight/day.

37. The method of claim 33, wherein the citrus is tangerines, oranges, lemons or grapefruits.

38. The method of claim 33, wherein the citrus peel extract is prepared by a process including the steps of: adding 3 to 30 l of 20 to 95% ethanol to 1 kg of dried citrus peel; allowing the mixture to stand at a temperature ranging from 25 to 80° C. for a period ranging from 1 to 12 hours; filtering the resulting extract; and concentrating the filtrate to obtain the citrus peel extract.

39. The method of claim 33, wherein the citrus peel extract is prepared by a process including the steps of: adding 5 to 30 l of 0.1 to 2% Ca(OH)₂ or NaOH to 1 kg of dried citrus peel; allowing the mixture to stand at a temperature ranging from 25 to 60° C. for a period ranging from 1 to 5 hours; filtering the resulting extract; adjusting the filtrate to a pH ranging from 4.0 to 7.0; allowing the resulting filtrate to stand at a temperature ranging from 1 to 10° C. for a period ranging from 10 to 48 hours; and, recovering and drying the resulting precipitate to obtain the citrus peel extract.

40. The method of claim 33, wherein the citrus peel powder is prepared by a process including the steps of: lyophilizing or drying the solid materials remaining after squeezing juice from citrus fruits; and powdering the dried materials to a particle size ranging from 50 to 250 μm.

41. The method of claim 33, wherein the citrus peel extract or citrus peel powder is administered in the form of a pharmaceutical composition containing an effective amount of the citrus peel extract and a pharmaceutically acceptable carrier.

42. The method of claim 33, wherein the citrus peel extract or citrus peel powder is administered in the form of an additive or a dietary supplement in food or beverage.

43. The method of claim 42, wherein the content of the citrus peel extract in the food ranges from 0.5 to 10% by weight.

44. The method of claim 42, wherein the content of the citrus peel powder in the food ranges from 1 to 30% by weight.

45. The method of claim 42, wherein the food is meats, chocolates, snacks, confectionery, pizza, foods made from cereal flour, gums, dairy products, soups, broths, pastes, ketchups, sauces, vitamin complexes or health foods.

46. The method of claim 45, wherein the foods made from cereal flour is breads, cakes, crackers, cookies, biscuits or noodles.

47. The method of claim 42, wherein the beverage is dairy products, vegetable juices, fruit juices, teas, alcoholic beverages or carbonated beverages.

48. The method of claim 42, wherein the content of the citrus peel extract in the beverage ranges from 10 to 100 g per 1,000 ml of the beverage.
